(19) United States
(12) Patent Application Publication
(10) Pub. No.: US 2024/0112389 A1
(43) Pub. Date: Apr. 4, 2024

(54) INTENTIONAL VIRTUAL USER EXPRESSIVENESS

(71) Applicant: Microsoft Technology Licensing, LLC, Redmond, WA (US)

(72) Inventors: Gino G. Buzzelli, New York, NY (US); Scott A. SCHWARZ, Seattle, WA (US)

(21) Appl. No.: 17/957,712

(22) Filed: Sep. 30, 2022

(51) Int. Cl. G06T 13/40; G06V 40/16

(52) U.S. Cl. CPC G06T 13/40; G06V 40/174

(57) ABSTRACT

A method and system for displaying an emotional states of a user using a graphical representation of the user are disclosed herein, including receiving a configuration instruction for a first emotional state, detecting an emotional state of the user using sentiment analysis, determining a modified emotional state for the graphical representation of the user based upon the detected emotional state of the user and the configuration instruction, selecting a rule from a set of facial animation rules based upon the modified emotional state and the detected emotional state of the user, and causing the graphical representation of the user to be rendered using the selected rule.

### TECHNICAL FIELD

The present disclosure generally refers to systems and methods for intentional virtual user expressiveness in accordance with some embodiments.

### BACKGROUND

The use of software and hardware technologies for meeting and communication services continues to increase as technology evolves. As technology evolves, online virtual presence in meetings will continue to grow. Facial recognition technologies have developed that can determine facial features of a person for various applications, such as personal identification or verification. In addition, facial emotions can be detected to enable richer meeting services for meeting and communication services.

### SUMMARY

Embodiments of the present disclosure include a method and system for displaying real-time emotional states of a user through a graphical representation of the user, such as during a communication session, an online gaming session, in a user profile, or in one or more sessions or situations where a real-time or near-real time graphical representation of the user is displayed. Configuration instructions for one or more emotional states can be received specifying that one or more detected emotional states of the user are to be modified. An emotional state of the user, and in certain examples, a magnitude of the emotional state, can be detected or determined based on a received image of the user, such as during a communication or other session, or other information received from or about the user during the communication or other session, using sentiment analysis. A modified emotional state for the graphical representation of the user can be determined based upon the detected emotional state of the user and the configuration instruction. The modified emotional state of the graphical representation can modify the detected emotional state by displaying a different emotional state or by changing a magnitude of the detected emotional state. A rule can be selected from a set of facial animation rules based upon the modified emotional state and the detected emotional state of the user specifying instructions for rendering a graphical representation of the user that has a facial expression that is mapped to the modified emotional state. The graphical representation of the user can be rendered using the selected rule.

The claimed subject matter may be implemented as a method, apparatus, or article of manufacture using standard programming and engineering techniques to produce software, firmware, hardware, or any combination thereof to control a computing device to implement the disclosed subject matter. The term, “article of manufacture,” as used herein is intended to encompass a computer program accessible from any computer-readable storage device or media. Computer-readable storage media can include, but are not limited to, magnetic storage devices, e.g., hard disk, floppy disk, magnetic strips, optical disk, compact disk (CD), digital versatile disk (DVD), smart cards, flash memory devices, among others. In contrast, computer-readable media (i.e., not storage media) may additionally include communication media such as transmission media for wireless signals, etc.

This Summary is provided to introduce a selection of concepts that are further described below in the Detailed Description. It is not intended to identify key features or essential features of the claimed subject matter, nor is it intended to be used to limit the scope of the claimed subject matter.

### BRIEF DESCRIPTION OF THE SEVERAL VIEWS OF THE DRAWINGS

The disclosed aspects will hereinafter be described in connection with the appended drawings that are provided to illustrate and not to limit the disclosed aspects.

FIG. 1 illustrates an example communication session between multiple participants having respective user devices executing respective meeting applications.

FIG. 2 illustrates example control areas and different first and second representations of a user at different meeting application settings.

FIG. 3 illustrates an example control area illustrating a radar chart adjustment feature providing different representations of a user at different meeting application settings.

FIGS. 4 and 5 illustrate example control areas including different meeting application adjustments.

FIG. 6 illustrates an example method for displaying an emotional state of a user using a graphical representation of the user during a communication session.

FIG. 7 illustrates an example method of determining a modified emotional state for the graphical representation of the user.

FIG. 8 illustrates an example method for determining a magnitude of an emotional state of the user.

FIG. 9 illustrates an example set of facial landmarks of a user.

FIG. 10 illustrates an example system for providing a graphical representations of a user in a networking environment.

### DETAILED DESCRIPTION

Meeting applications provide, over a data connection to a meeting or animation service, rich meeting service features, such as video, file or screen sharing, application sharing, document collaboration, rich messaging, access to corporate directories, group sharing and collaboration, or one or more other meeting application features. The meeting service can be connected to a meeting application through one or more data connections and configured to provide additional meeting services, such as transcription, captioning of shared video content, or one or more other meeting service features.

Meeting services and meeting applications additionally provide users control over how they are presented and perceived by others, such as by substituting, concealing, or obfuscating camera backgrounds, or providing customizable avatars that mimic their actions and emotions.

The present inventors have recognized, among other things, that non-verbal communication or visual expression of a graphical representation of the user can be determined, then disabled, suppressed, or modified in a predefined way, such as to encourage participation in communication or other sessions, display of profile representations, etc., without unknowingly or unintentionally communicating negative or undesired emotions to the audience, to provide a desired emotional response instead of the determined emotional response, or to modify a determined emotional response. In certain examples, the user can provide configuration instructions for specific emotional states, such as to suppress or disable specific determined emotional states, replacing specific determined emotional states with one or more other emotional states, or modifying a magnitude of the determined emotional state, such as when rendering the graphical representation of the user.

A technical solution contemplated herein provides, in certain examples, customization and control of a graphical representation of the user during communication or other sessions, in a profile representation, etc., to address the technical problem of data privacy and security, obfuscating and protecting unintentionally communicated information in specific settings or situations by selecting one or more rules from a set of facial animation rules and rendering graphical representations of the user mapped to a modified emotional state according to the selected one or more rules.

Graphical representations of the user can include avatar representations of the user, such as determined and specified or configured by the user, or automatically provided to the user by meeting or animation services, such as based on an image of the user. In other examples, graphical representations of the user can include live video or static images of the user, in certain examples selected or modified to provide desired emotional states.

Rendering modified emotional states of the user can include rendering avatar representations of the user, or in certain examples, providing deepfake representations of the user perceived as a live image to an audience, with a modified emotional state than that determined or detected from the user, such as using an artificial intelligence (AI) deep learning model, locally by a user device or remotely by a meeting service or an animation service (e.g., as an API call, etc.). A facial animation model executed by one or more processors, such as of the user device, the meeting service, or the animation service, can render a modified representation of the user according to one or more of a set of facial animation rules, selected and loaded for rendering according to a determined emotional state of the user and a configuration instruction for the determined emotional state. Avatar representations of the user or modified live representations of the user can be referred to as synthetic media or synthetic representations. However, in certain examples, depending on the configuration instructions and the determined emotional state of the user, an unmodified live video or image of the user can be provided for display, such as in the communication or other session, in a profile representation, etc., for example, when the configuration instructions are silent as to changes or modifications to a specific emotional state, or actively instruct the systems or methods to provide a detected or unmodified representation of the user.

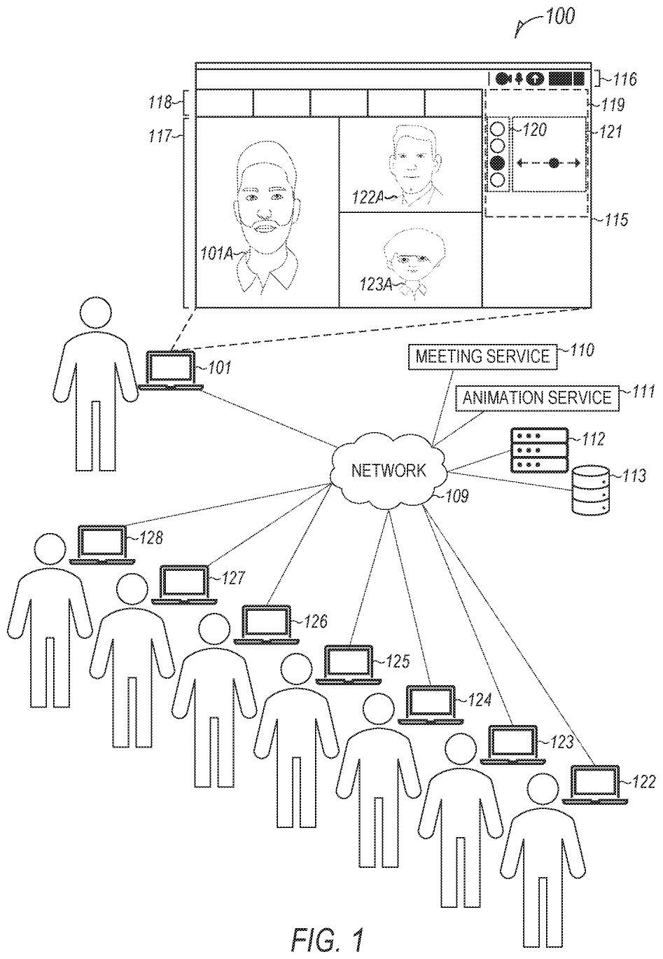

FIG. 1 illustrates an example communication session 100 between multiple participants having respective user devices executing respective meeting applications coupled to a meeting service 110 or an animation service 111 through a network 109. The meeting service 110 or the animation service 111 can include or otherwise be coupled to one or more servers or databases, such as a server 112 or a database 113, such as to store or process user information or provide one or more services associated with the communication session, including presentation of the communication session 100. In certain examples, the animation service 111 can be a component of the meeting service 110. In other examples, the meeting service 110 can be separate from but coupled to the animation service 111.

The communication session 100 includes a first user, such as a host or a presenter, coupled to a first user device 101 executing a meeting application and sharing information in a first portion 117 of a meeting application view 115, such as a representation of the first user 101A with or without accompanying audio data of the first user or other shared information (e.g., a video stream of an active portion of an active screen of the first user device 101, one or more applications executed on the first device 101, a document, etc.), and providing such information to the meeting service 110 or the animation service 111 for management and display, over the network 109, to multiple second user devices 122-128 of multiple participants or audience members, each executing a meeting application connected to the meeting service 110. The communication session 100 further optionally includes representations of one or more other users, such as representations of a second user 122A and a third user 123A in the first portion 117 of the meeting application view 115, and optionally other participants or audience members in a second portion 118 of the meeting application view 115. The first and second portions 117, 118 of the meeting application view 115 can change sizes and organization depending on, for example, active participants of the communication session 100, participants sharing audio or a representation or video feed in the communication session 100, hosts of the communication session 100, etc.

In an example, the meeting application view 115 can optionally include one or more controls 116 for the meeting application, and a feature area selectively providing different communication session features, such as a transcript box to show live transcribed text, a list of participating or invited users, a message (or chat) box for user discussion, or settings or configurations for a respective user from the perspective of the user or for host control of other users. In FIG. 1, the feature area includes a control area 119 for the representation of the user. The control area 119 can include various controls inputs, such as select boxes, etc., configured to control various aspects of the displayed representation, such as selectively displaying the representation of the user as an avatar, a live video feed, or a static image or icon, as well as various controls including those to modify one or more emotional states or emotional responses of the representation of the user. The control area 119 can include select icons 120 configured to turn on or off various emotions, and adjustment features 121 for respective selected icons. For example, the meeting application can receive input from a user unselecting display of specific emotions, such as anger or sadness, etc. In other examples, the meeting application can receive adjustment of one or more specific emotions, such as increasing or decreasing a response of a specific emotion, such as surprise or happiness, etc. One or more of the meeting service 110, the animation service 111, or the meeting application can be configured to modify a detected emotional response of the user based on the received selections and render the representation of the user based on the modified emotional response.

Although illustrated herein with respect to a communication session, such customization and control of the representation of the user is applicable in other sessions or situations including display of a graphical representation of the user.

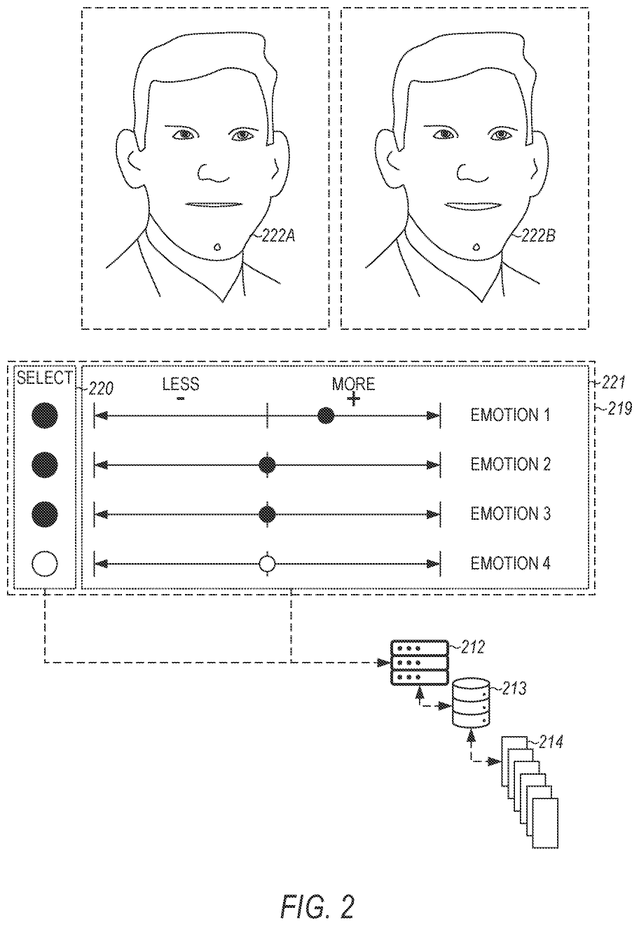

FIG. 2 illustrates example control areas 219 and different first and second representations 222A, 222B of a user (e.g., the second user 122A of FIG. 1) at different meeting application settings, such as illustrated by select icons 220 and adjustment features 221 (e.g., one or more sliders, etc.) of the control area 219 of a meeting application. The meeting application can provide the different meeting application settings as configuration instructions to one or more meeting or animation service, or components of the meeting or animation service, such as a server 212 or a database 213 connected or available to the meeting or animation service, for example, to select one or more rules 214 for rendering the representation of the user.

For example, the first representation of the user 222A can be a live video version of the user or an avatar representation mimicking one or more emotions, actions, or responses of the user. The select icons 220 in FIG. 2 include four emotions (e.g., happiness, sadness, surprise, anger, etc.). Emotions 1-3 are selected. Emotion 4 is not selected (e.g., anger). Each adjustment of the adjustment features 221 includes a central mark (e.g., normal response) with less and more adjustments at the left and right, respectively. Emotion I (e.g., happiness) has been adjusted from the central mark to be more. The second representation of the user 222B is a representation of the user with more happiness response, such as rendered by one or more of the meeting application, the meeting service, or the animation service.

In certain examples, the meeting application can provide one or more of the first and second representations 222B, 222C to the user as preview images before providing the rendered representation of the user to the one or more other participants or audience members of a communication session, etc.

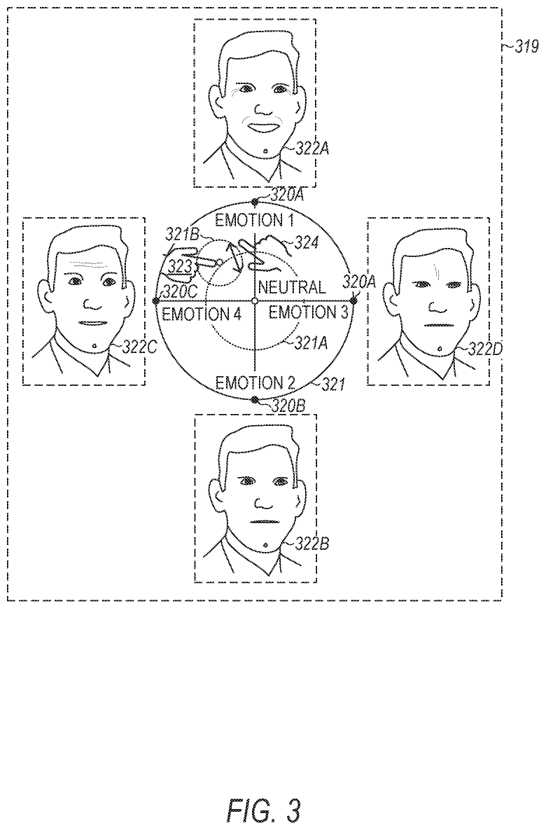

FIG. 3 illustrates an example control area 319 illustrating a radar chart adjustment feature 321 providing different representations 322A-322D of a user (e.g., the second user 122A of FIG. 1) at different meeting application settings, such as illustrated by select icons 320A-320D and adjustment features 321A, 321B of the control area 319 of a meeting application. In certain examples, select icons 320A-320D can be selected or unselected, making different segments or portions of the radar chart available or unavailable. Additionally, first and second selected adjustment features 321A, 321B can be determined, such as by dragging a point of an adjustment feature (e.g., using a first input 323), or adjusting a size of the adjustment feature, such as by pinching or expanding the adjustment feature (e.g., using a second input 324).

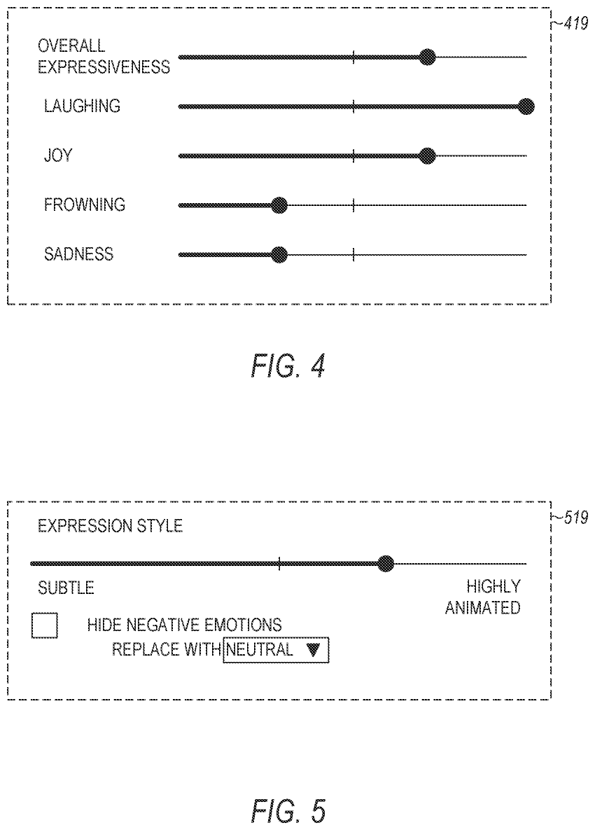

FIGS. 4 and 5 illustrate example control areas 419, 519, including different meeting application adjustments. For example, the control area 419 can include separate adjustments of different emotions greater than (to the right of) or less than (to the left of) an origin (e.g., unmodified response) illustrated using a vertical mark, including overall expressiveness (e.g., controlling a magnitude of a modified emotional response), and individual emotions (e.g., laughing, joy, frowning, sadness, etc.). The control area 519 includes a single slide adjustment configured to increase or decrease one or more detected emotions, and an optional selection box to hide negative emotions (e.g., one or more of sadness, anger, etc.). In certain examples, negative emotions can be replaced with a neutral response, or one or more other responses, such as illustrated by the drop-down selection, for example, replaced with a previously detected or displayed emotion, or replaced with a specific selected emotion, etc. In other examples, one or more other selection boxes or replacement drop-down selections can be included.

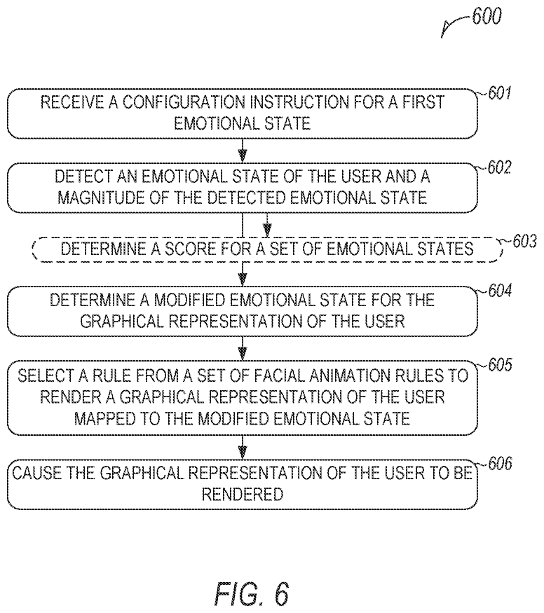

FIG. 6 illustrates an example method 600 for displaying an emotional state of a user using a graphical representation of the user, the graphical representation having a displayed emotional state, in certain examples, corresponding to a real-time emotional state of the user. At step 601, a configuration instruction for a first emotional state can be received, such as from a user profile or a user database, or from one or more inputs from a user, such as through a user interface of a meeting application. For example, through user interfaces from FIGS. 2-5. In an example, the configuration instruction can specify that the first emotional state is to be modified, such as to enhance or diminish the first emotional state, to instead provide one or more other emotional states, to carry forward a previously detected emotional state (e.g., a preceding emotional state), to provide a neutral emotional state, etc.

In an example, the first emotion can be a negative emotion, such as sadness, disgust, or anger. In other examples, the first emotion can be one of a larger set of emotions. In certain examples, configuration instructions for one or more other emotional states can be received, separately or in combination (e.g., a configuration instruction to hide negative emotions, to enhance positive emotions, etc.). In certain examples, the set of emotions can include happiness, sadness, neutral, anger, contempt, disgust, surprise, fear, etc.

At step 602, an emotional state of the user can be detected, such as using an image or a video feed of the user and sentiment analysis of the image or the video feed. In certain examples, a magnitude of the emotional state can be detected, such as using the sentiment analysis, for example, by analyzing one or more features of a face of the user, a voice of the user, a posture of the user, or one or more other motions or reactions of the user. In an example, sentiment analysis of the user can be performed using the meeting application, the meeting service, or the animation service. In some examples, a Convolutional Neural Network (CNN) or Support Vector Machine (SVM) are used to determine the emotional state of the user. In some examples, supervised machine-learning models such as the CNN or SVM utilize training data sets of facial images labeled with the user's emotions. In some examples, the models may operate on static images. In these examples, a still image of the video from the user may be sampled periodically (e.g., every 1 second) to approximate the user's emotions during the video.

At step 603, a score for a set of emotional states can optionally be determined by the sentiment analysis, such as to determine the detected emotional state of the user, for example, as the emotional state from the set of emotional states having the highest determined score. A received image of the user can be analyzed and scored against emotional templates, determined using information from the user or from a number of users. In certain examples, the emotion having the highest score can be selected as the detected emotional state of the user.

At step 604, a modified emotional state for the graphical representation of the user can be determined, such as using one or more of the meeting application, the meeting service, or the animation service, based upon the detected emotional state of the user and the configuration instructions. The modified emotional state of the graphical representation can modify the detected emotional state by being a different emotional state or a change in the detected magnitude of the detected emotional state. In certain examples, the magnitude of the detected emotional state can be commensurate with a determined score, relative to a population of determined scores or determined scores of the user, a set of emotional classification rules based on user or population information (e.g., training data, etc.), etc. For example, if a configuration instruction instructs the system to amplify detected emotions, and the determined emotional score is a 50 on a scale between 0 and 100, the modified emotional state can be increased to be greater than 50, etc.

At step 605, a rule can be selected from a set of facial animation rules, such as using one or more of the meeting application, the meeting service, or the animation service, based upon, for example, the modified emotional state and the detected emotional state of the user. The selected rule, or the set of facial animations rules, specify instructions to set parameters for rendering the graphical representation of the user that has a facial expression that is mapped to the modified emotional state. For example, detected or determined facial features can be adjusted to the one or more mapped or modified emotional states, such as from a detected or received image or representation of the user.

In certain examples, the selected rules and the amount of modification is dependent not only on the perceived or determined emotional state of the user, but also the determined magnitude of the emotional state of the user. In an example, the magnitude of the modified emotional state can be determined according to the determined magnitude of the determined emotional state of the user, in certain examples, modified by the received configuration instructions. In other examples, the facial animation rules can include one or more weights for a deep reinforcement machine-learning model, such as to increase or decrease a magnitude of a modified emotional state of the user. That is, the model may take an image of the user and a desired magnitude of an emotion to produce a modified image. In some examples, the facial animation rules may include an autoencoder neural network. The autoencoder may include a generative adversarial network.

At step 606, one or more of the meeting application, the meeting service, or the animation service can cause the graphical representation of the user to be rendered, for example, mapped to the modified emotional state, and the rendered graphical representation of the user can be displayed to one or more participants or audience members of a communication or other session, etc., such as on a meeting application executed on a user device and connected to the meeting service.

Although described herein with respect to image data, in other examples, audio data can be received and analyzed to determine sounds or tone associated with an emotion, such as separate from any received image information, or as a supplement to the received image information.

In certain examples, analyzing data to determine facial detection and expressions, training one or more animation models and rendering the graphical representation of the user can be performed, such as disclosed in the commonly assigned Mittal et al. U.S. Pat. No. 11,238,885 titled “COMPUTING SYSTEM FOR EXPRESSIVE THREE-DIMENSIONAL FACIAL ANIMATION,” Koukoumidis et al. U.S. Pat. No. 10,878,307 titled “EQ-DIGITAL CONVERSATION ASSISTANT,” Xu et al. U.S. patent application Ser. No. 12/950,801 titled “REAL-TIME ANIMATION FOR AN EXPRESSIVE AVATAR,” each of which are incorporated herein in their entireties, including their description of animating a visual representation of a face of the user, training animation models, etc.

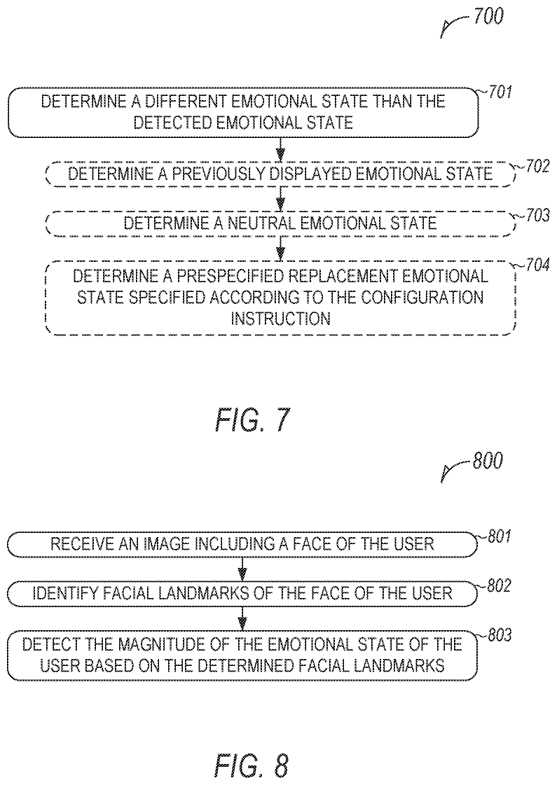

FIG. 7 illustrates an example method 700 of determining a modified emotional state for the graphical representation of the user. At step 701, a different emotional state than the detected emotional state can be determined, such as using one or more of the meeting application, the meeting service, or the animation service, based upon the detected emotional state of the user and the configuration instructions.

At step 702, the modified emotional state can include a previously displayed emotional state. At step 703, the modified emotional state can include a neutral emotional state. At step 704, the modified emotional state can include a prespecified replacement emotional state specified according to a received configuration instruction. For example, a user can provide, in one or more configuration instructions, replacement emotions for one or more specific emotions (e.g., replace sadness with happiness, etc.).

FIG. 8 illustrates an example method 800 for determining a magnitude of an emotional state of the user. At step 801, an image can be received, such as from a meeting application executed on a user device, including a face of the user. In certain examples, the face of the user can be detected, such as by one or more of a meeting application, the meeting service, or the animation service, such as through image processing techniques, for example, through a neural network based on trained image data.

At step 802, facial landmarks, features, or attributes of the face of the user can be identified, for example, to identify the user, or to determine the emotional state of the user based on one or more identified facial reactions or changes or movement of one or more facial landmarks or features.

At step 803, the magnitude of the emotional state of the user can be detected based on the determined facial landmarks. In certain examples, information from the determined emotional states can be used to create a model or changes in the facial landmarks or features of the face, such as to render a modified emotional state of the user. Movement of or changes in the facial landmarks or features can be used to determine one or more rules of the set of facial animation rules, such as to modify or render the graphical representation of the user.

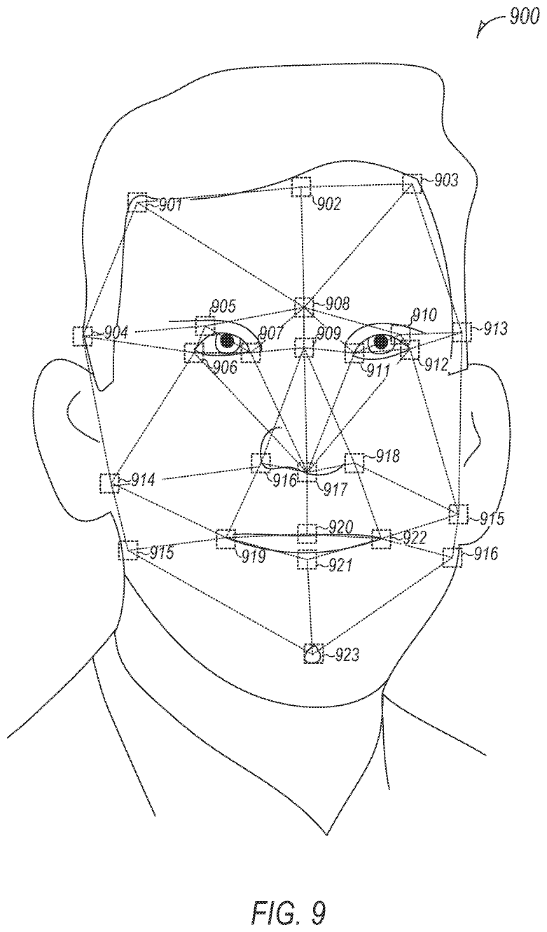

FIG. 9 illustrates an example set of facial landmarks 901-923 of a user 900 (e.g., the second user 122A of FIG. 1), for example, determined by one or more of a meeting application, a meeting service, or an animation service, such as by edge detection, etc. Facial landmarks 901-923 or features, including measurements of distance, area, shape, or one or more other measurements of or between respective landmarks, can be determined for a respective image, or across a number of images occurring near the same time, such as to identify an emotional response of the user. Determined facial landmarks can include, among other things, one or more of locations of pupils of the user, boundaries of eyes with respect to eyebrows, smiling marks, detection of smile lines, the corners or top and bottom of a mouth, hairline features, ear locations, nose features (e.g., a tip of the nose, edges of the nose, etc.), etc.

In an example, sentiment analysis can use one or more of the determined facial landmarks 901-923 or measurements to detect an emotion of the user, such as using a facial action coding system (FACS), etc. In other examples, one or more deep learning systems, such as a neural network, a convolutional neural network, a deep reinforcement machine-learning model etc., can be used to determine one or more of the facial landmarks 901-923 or detect the emotion of the user. In certain examples, one or more of the detected facial landmarks can be adjusted or moved during rendering, replacing or morphing the representation of the user.

In certain examples, respective landmarks can be used to create one or more training models, facial animation models, or facial animation rules to render a modified emotional response on the representation of the user. For example, measurements can include relative distances between specific facial landmarks (e.g., a grouping around an eye, such as facial landmarks 905-907 or 910-912, etc.), determined shapes of specific facial landmarks, or changes in specific landmarks, etc.

Specific sets of facial animation rules can be determined, for example, using relative changes between specific facial landmarks 901-923 of a user between detected emotional states. In other examples, relative changes in determined facial landmarks from data from one or more other users can be used to determine the set of facial animation rules. For example, changing a detected emotional response from happy to sad can include determining a shape of a mouth of the user in the sad state and implementing a set of movements of specific facial landmarks of the mouth to modify the mouth into the different shape.

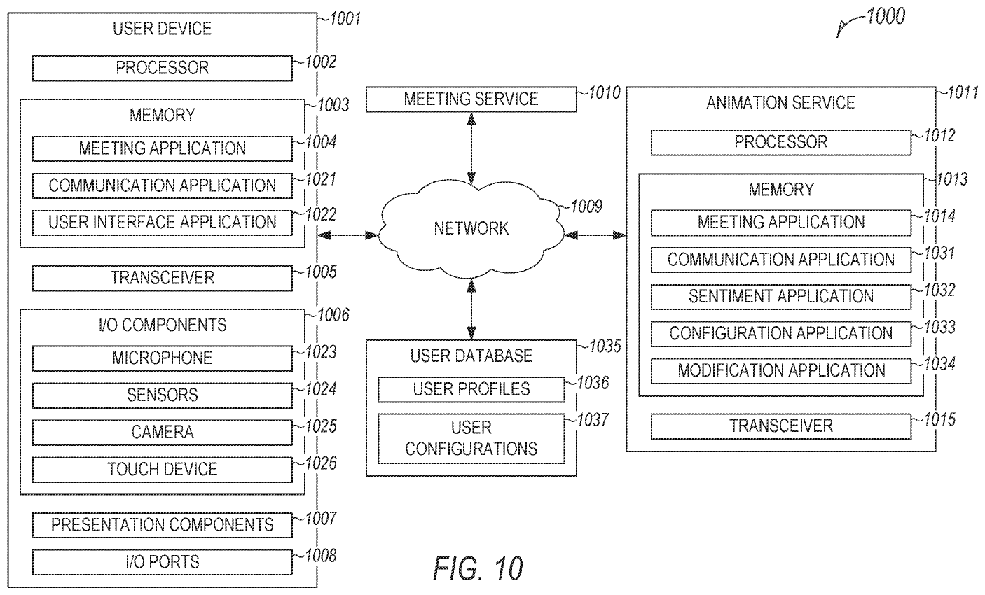

FIG. 10 illustrates an example system 1000 for providing a graphical representations of a user during a communication session including a first user device 1001 in a networking environment including a meeting service 1010, an animation service 1011, and a user database 1035 communicating over a network 1009. In certain examples, the first user device 1001 is exemplary and the system 1000 can include one or more other user devices (e.g., a second user device, etc.).

The first user device 1001 can include a processor 1002 (e.g., one or more processors), a memory 1003, a transceiver 1005, input/output (I/O) components 1006, one or more presentation components 1007, and one or more I/O ports 1008. The first user device 1001 can take the form of a mobile computing device or any other portable device, such as a mobile telephone, laptop, tablet, computing pad, notebook, gaming device, portable media player, etc. in other examples, the first user device 1001 can include a less portable device, such as desktop personal computer, kiosk, tabletop device, industrial control device, etc. Other examples can incorporate the first user device 1001 as part of a multi-device system in which two separate physical devices share or otherwise provide access to the illustrated components of the first user device 1001.

The processor 1002 can include any quantity of processing units and is programmed to execute computer-executable instructions for implementing aspects of the disclosure. The instructions may be performed by the processor or by multiple processors within the computing device or performed by a processor external to the first user device 1001. In some examples, the processor 1002 is programmed to execute methods, such as the one or more methods illustrated herein, etc. Additionally, or alternatively, the processor 1002 can be programmed to present an experience in a user interface (“UI”). For example, the processor 1002 can represent an implementation of techniques to perform the operations described herein.

The memory 1003 can include a meeting application 1004, in certain examples configured to interact with or connect to the meeting service 1010 or the animation service 1011. While the meeting application 1004 can be executed on the first user device 1001 (or one or more other user devices), the meeting service 1010 and the animation service 1011 can include separate services separate and remote from the first user device 1001, and can include a server, network, or cloud-based services accessible over the network 1009.

In an example, the meeting application 1004 can include a local client, such as a Microsoft Teams client, a Skype client, etc., installed on a respective user device and connected to the meeting service 1010, such as a cloud-based meeting service or platform (e.g., Microsoft Teams, Skype, etc.), or the animation service 1011. In other examples, the meeting application 1004 can include a virtual application (e.g., a network-, web-, server-, or cloud-based application) accessing resources of a respective user device, or combinations of a local client and a virtual application, etc.

The animation service 1011 can be exemplary of one or more other services, such as the meeting service 1010. One or both of the animation service 1011 and the meeting service 1010 can manage a communication session, including communication streams, such as emails, documents, chats, comments, texts, images, animations, hyperlinks, or voice or video communication for users associated with one or more online or other communication sessions through meeting applications executed on connected devices, such as the first user device 1001 or one or more other devices including hardware and software configured to enable meeting applications or one or more other communication platforms to communicate to or from the respective devices.

The transceiver 1005 can include an antenna capable of transmitting and receiving radio frequency (“R”) signals and various antenna and corresponding chipsets to provide communicative capabilities between the first user device 1001 and one or more other remote devices. Examples are not limited to RF signaling, however, as various other communication modalities may alternatively be used.

The presentation components 1007 can include, without limitation, computer monitors, televisions, projectors, touch screens, phone displays, tablet displays, wearable device screens, televisions, speakers, vibrating devices, and any other devices configured to display, verbally communicate, or otherwise indicate image search results to a user of the first user device 1001 or provide information visibly or audibly on the first user device 1001. For example, the first user device 1001 can include a smart phone or a mobile tablet including speakers capable of playing audible search results to the user. In other examples, the first user device 1001 can include a computer in a car that audibly presents search responses through a car speaker system, visually presents search responses on display screens in the car (e.g., situated in the car's dashboard, within headrests, on a drop-down screen, etc.), or combinations thereof. Other examples present the disclosed search responses through various other display or audio presentation components 1007.

I/O ports 1008 allow the first user device 1001 to be logically coupled to other devices and I/O components 1006, some of which may be built into the first user device 1001 while others may be external. I/O components 1006 can include a microphone 1023, one or more sensors 1024, a camera 1025, and a touch device 1026. The microphone 1023 can capture speech from the user and/or speech of or by the user. The sensors 1024 can include any number of sensors on or in a mobile computing device, electronic toy, gaining console, wearable device, television, vehicle, or other device, such as one or more of an accelerometer, magnetometer, pressure sensor, photometer, thermometer, global positioning system (“GPs”) chip or circuitry, bar scanner, biometric scanner for scanning fingerprint, palm print, blood, eye, or the like, gyroscope, near-field communication (“NFC”) receiver, or any other sensor configured to capture data from the user or the environment. The camera 1025 can capture images or video of or by the user. The touch device 1026 can include a touchpad, track pad, touch screen, or other touch-capturing device. In other examples, the i/O components 1006 can include one or more of a sound card, a vibrating device, a scanner, a printer, a wireless communication device, or any other component for capturing information related to the user or the environment.

The memory 1003 can include any quantity of memory associated with or accessible by the first user device 1001. The memory 1003 can be internal to the first user device 1001, external to the first user device 1001, or a combination thereof. The memory 1003 can include, without limitation, random access memory (RAM), read only memory (ROM), electronically erasable programmable read only memory (EEPROM), flash memory or other memory technologies, CDROM, digital versatile disks (DVDs) or other optical or holographic media, magnetic cassettes, magnetic tape, magnetic disk storage or other magnetic storage devices, memory wired into an analog computing device, or any other medium for encoding desired information and for access by the first user device 1001. The terms computer-readable medium, machine readable medium, and storage device do not include carrier waves to the extent carrier waves are deemed too transitory. The memory 1003 can take the form of volatile and/or nonvolatile memory, can be removable, non-removable, or a combination thereof; and can include various hardware devices, e.g., solid-state memory, hard drives, optical-disc drives, etc. Additionally, or alternatively, the memory 1003 can be distributed across multiple user devices, such as in a virtualized environment in which instruction processing is carried out on multiple ones of the first user device 1001. The memory 1003 can store, among other data, various device applications that, when executed by the processor 1002, operate to perform functionality on the first user device 1001. Example applications can include search applications, instant messaging applications, electronic-mail application programs, web browsers, calendar application programs, address book application programs, messaging programs, media applications, location-based services, search programs, and the like. The applications may communicate with counterpart applications or services such as web services accessible via the network 1009. For example, the applications can include client-operating applications that correspond to server-side applications executing on remote servers or computing devices in the cloud.

Instructions stored in the memory 1003 can include, among other things, one or more of a meeting application 1004, a communication application 1021, and a user interface application 1022 executed on the first user device 1001. The communication application 1021 can include one or more of computer-executable instructions for operating a network interface card and a driver for operating the network interface card. Communication between the first user device 1001 and other devices can occur using any protocol or mechanism over a wired or wireless connection or across the network 1009. In some examples, the communication application 1021 is operable with RF and short-range communication technologies using electronic tags, such as NFC tags, Bluetooth® brand tags, etc.

In some examples, the user interface application 1022 includes a graphics application for displaying data to the user and receiving data from the user. The user interface application 1022 can include computer-executable instructions for operating the graphics card to display search results and corresponding images or speech on or through the presentation components 1007. The user interface application 1022 can interact with the various sensors 1024 and camera 1025 to both capture and present information through the presentation components 1007.

One or both of the meeting service 1010 and the animation service 1011 can be configured to receive user and environment data, such as received from the first user device 1001 or one or more other devices over the network 1009. In certain examples, the meeting service 1010 or the animation service 1011 can include one or more servers, memory, databases, or processors, configured to execute different web-service computer-executable instructions, and can be configured to provide and manage one or more meeting services for one or more users or groups of users, such as users of the first user device 1001 or one or more other devices. The animation service 1011 can be capable of providing and receiving messages or other information including images, videos, audio, text, and other communication media to or from the first user device 1001 or one or more other devices over the network 1009.

The networking environment illustrated in FIG. 10 is an example of one suitable computing system environment and is not intended to suggest any limitation as to the scope of use or functionality of examples disclosed herein. The illustrated networking environment should not be interpreted as having any dependency or requirement related to any single component, module, index, or combination thereof, and in other examples, other network environments are contemplated.

The network 1009 can include the internet, a private network, a local area network (LAN), a wide area network (WAN), or any other computer network, including various network interfaces, adapters, modems, and other networking devices for communicatively connecting the first user device 1001, the meeting service 1010, and the animation service 1011. The network 1009 can also include configurations for point-to-point connections.

The animation service 1011 includes a processor 1012 to process executable instructions, a memory 1013 embodied with executable instructions, and a transceiver 1015 to communicate over the network 1009. The memory 1013 can include one or more of: a meeting application 1014, a communication application 1031, a sentiment application 1032, a configuration application 1033, a modification application 1034, or one or more other applications, modules, or devices, etc. While the animation service 1011 is illustrated as a single box, it is not so limited, and can be scalable. For example, the animation service 1011 can include multiple servers operating various portions of software that collectively generate composite icons or templates for users of the first user device 1001 or one or more other devices.

The user database 1035 can provide backend storage of Web, user, and environment data that can be accessed over the network 1009 by the animation service 1011 or the first user device 1001 and used by the animation service 1011 to combine subsequent data in a communication stream. The Web, user, and environment data stored in the database includes, for example but without limitation, one or more user profiles 1036 and user configurations 1037. In certain examples, the user configurations 1037 can include different configurations dependent at least in part on other participants of a meeting or audience of the animation. Additionally, though not shown for the sake of clarity, the servers of the user database 1035 can include their own processors, transceivers, and memory. Also, the networking environment depicts the user database 1035 as a collection of separate devices from the animation service 1011. However, examples can store the discussed Web, user, configuration, and environment data shown in the user database 1035 on the animation service 1011 or the meeting service 1010.

The user profiles 536 can include electronically stored collection of information related to the user. Such information can be stored based on a user's explicit agreement or “opt-in” to having such personal information be stored, the information including the user's name, age, gender, height, weight, demographics, current location, residency, citizenship, family, friends, schooling, occupation, hobbies, skills, interests, Web searches, health information, birthday, anniversary, celebrated holidays, moods, user's condition, and any other personalized information associated with the user. The user profile includes static profile elements, e.g., name, birthplace, etc., and dynamic profile elements that change over time, e.g., residency, age, condition, etc.

Additionally, the user profiles 536 can include static and/or dynamic data parameters for individual users. Examples of user profile data include, without limitation, a user's age, gender, race, name, location, interests, Web search history, social media connections and interactions, purchase history, routine behavior, jobs, or virtually any unique data points specific to the user. The user profiles 536 can be expanded to encompass various other aspects of the user.

The present disclosure relates to systems and methods for triggering hand-off of an incoming call event from a default first telephony application to a second telephony application on a device according to at least the examples provided in the sections below:

(A1) In one aspect, some embodiments or examples include displaying an emotional state of a user using a graphical representation of the user, the graphical representation having a displayed emotional state, the displaying comprising receiving a configuration instruction for a first emotional state, the configuration instruction specifying that the first emotional state is to be modified, detecting, based on a received image of the user, an emotional state of the user and a magnitude of the detected emotional state of the user using sentiment analysis, determining a modified emotional state corresponding to the detected emotional state for the graphical representation of the user based upon the detected emotional state of the user and the configuration instruction, the modified emotional state of the graphical representation modifying the detected emotional state by being a different emotional state or a change in the magnitude of the detected emotional state, selecting a rule from a set of facial animation rules based upon the modified emotional state and the detected emotional state of the user, the rule specifying instructions for rendering the graphical representation of the user that has a facial expression that is mapped to the modified emotional state, and causing the graphical representation of the user to be rendered using the selected rule.

(A2) In some embodiments of A1, determining the modified emotional state for the graphical representation of the user based upon the detected emotional state of the user and the configuration instruction comprises determining a different emotional state than the detected emotional state, including one of a previously displayed emotional state of the user preceding the determined emotional state, a neutral emotional state, or a prespecified replacement emotional state specified according to the configuration instruction.

(A3) In some embodiments of A1-A2, receiving the configuration instruction for the first emotional state includes receiving the replacement emotional state.

(A4) In some embodiments of A1-A3, the first emotional state is one of a set of emotional states comprising happiness, sadness, neutral, anger, contempt, disgust, surprise, and fear, and detecting the magnitude of the detected emotional state of the user includes determining a score for the set of emotional states based on the received image of the user, and selecting the detected emotional state from the set of emotional states having a highest score as the detected emotional state of the user.

(A5) In some embodiments of A1-A4, detecting the magnitude of the detected emotional state of the user using sentiment analysis includes receiving the image including a face of the user, identifying facial landmarks of the face of the user from the received image, including locations of pupils of the user, a tip of a nose of the user, and a mouth of the user, and detecting the magnitude of the emotional state of the user based on the identified facial landmarks and a set of emotional classification rules.

(A6) In some embodiments of A1-A5, determining the magnitude of the emotional state of the user comprises determining facial attributes of the user based on one or more of the identified facial landmarks, the facial attributes including measurements of one or more of the identified facial landmarks or between two or more of the identified facial landmarks and determining the magnitude of the emotional state of the user based on the determined facial attributes.

(A7) In some embodiments of A1-A6, causing the graphical representation of the user to be rendered using the selected rule comprises generating an avatar representation of the user for display.

(A8) In some embodiments of A1-A7, receiving the configuration instruction includes receiving an input from the user to suppress the first emotional state or to modify a magnitude of the first emotional state of the user from a default or previously received configuration instruction.

(A9) In some embodiments of A1-A8, causing the graphical representation of the user to be rendered using the selected rule comprises rendering a synthetic image of the user to communicate the displayed emotional state using a facial animation model, wherein the facial animation model includes a training model of the user.

(A10) In some embodiments of A1-A9, the sentiment analysis comprises a neural network.

(A11) In some embodiments of A1-A10, the facial animation rules comprise one or more weights for a deep reinforcement machine-learning model.

In yet another aspect, some embodiments include a system including a processor and a memory device coupled to the processor and having a program stored thereon for execution by the processor to perform operations comprising any of the embodiments of A1-A11 described above in various combinations or permutations. In yet another aspect, some embodiments include a non-transitory computer-readable storage medium storing one or more programs for execution by one or more processors of a storage device, the one or more programs including instructions for performing any of the embodiments of A1-A11 described above in various combinations or permutations. In yet another aspect, some embodiments include a method or a system including means for performing any of the embodiments of A1-A11 described above in various combinations or permutations.

Although a few embodiments have been described in detail above, other modifications are possible. For example, the logic flows depicted in the figures do not require the particular order shown, or sequential order, to achieve desirable results. Other steps may be provided, or steps may be eliminated, from the described flows, and other components may be added to, or removed from, the described systems. Other embodiments may be within the scope of the following claims.

In the description herein, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The included description of example embodiments is, therefore, not to be taken in a limited sense, and the scope of the present invention is defined by the appended claims.

The functionality can be configured to perform an operation using, for instance, software, hardware, firmware, or the like. For example, the phrase “configured to” can refer to a logic circuit structure of a hardware element that is to implement the associated functionality. The phrase “configured to” can also refer to a logic circuit structure of a hardware element that is to implement the coding design of associated functionality of firmware or software. The term “module” refers to a structural element that can be implemented using any suitable hardware (e.g., a processor, among others), software (e.g., an application, among others), firmware, or any combination of hardware, software, and firmware. The term, “logic” encompasses any functionality for performing a task. For instance, each operation illustrated in the flowcharts corresponds to logic for performing that operation. An operation can be performed using, software, hardware, firmware, or the like. The terms, “component,” “system,” and the like may refer to computer-related entities, hardware, and software in execution, firmware, or combination thereof. A component may be a process running on a processor, an object, an executable, a program, a function, a subroutine, a computer, or a combination of software and hardware. The term, “processor,” may refer to a hardware component, such as a processing unit of a computer system.

Furthermore, the claimed subject matter may be implemented as a method, apparatus, or article of manufacture using standard programming and engineering techniques to produce software, firmware, hardware, or any combination thereof to control a computing device to implement the disclosed subject matter. The term, “article of manufacture,” as used herein is intended to encompass a computer program accessible from any computer-readable storage device or media. Computer-readable storage media can include, but are not limited to, magnetic storage devices, e.g., hard disk, floppy disk, magnetic strips, optical disk, compact disk (CD), digital versatile disk (DVD), smart cards, flash memory devices, among others. In contrast, computer-readable media, i.e., not storage media, may additionally include communication media such as transmission media for wireless signals and the like.

What is claimed is:

1. A method for displaying an emotional state of a user using a graphical representation of the user, the graphical representation having a displayed emotional state, the method comprising:
receiving a configuration instruction for a first emotional state, the configuration instruction specifying that the first emotional state is to be modified;
detecting, based on a received image of the user, an emotional state of the user and a magnitude of the detected emotional state of the user using sentiment analysis;
determining a modified emotional state corresponding to the detected emotional state for the graphical representation of the user based upon the detected emotional state of the user and the configuration instruction, the modified emotional state of the graphical representation modifying the detected emotional state by being a different emotional state or a change in the magnitude of the detected emotional state;
selecting a rule from a set of facial animation rules based upon the modified emotional state and the detected emotional state of the user, the rule specifying instructions for rendering the graphical representation of the user that has a facial expression that is mapped to the modified emotional state; and
causing the graphical representation of the user to be rendered using the selected rule.

2. The method of claim 1,
wherein determining the modified emotional state for the graphical representation of the user based upon the detected emotional state of the user and the configuration instruction comprises determining a different emotional state than the detected emotional state, including one of:
a previously displayed emotional state of the user preceding the determined emotional state;
a neutral emotional state; or
a prespecified replacement emotional state specified according to the configuration instruction.

3. The method of claim 2,
wherein receiving the configuration instruction for the first emotional state includes receiving the replacement emotional state.

4. The method of claim 1,
wherein the first emotional state is one of a set of emotional states comprising happiness, sadness, neutral, anger, contempt, disgust, surprise, and fear, and
wherein detecting the magnitude of the detected emotional state of the user includes determining a score for the set of emotional states based on the received image of the user and selecting the detected emotional state from the set of emotional states having a highest score as the detected emotional state of the user.

5. The method of claim 1,
wherein detecting the magnitude of the detected emotional state of the user using sentiment analysis includes:
receiving the image including a face of the user;
identifying facial landmarks of the face of the user from the received image, including locations of pupils of the user, a tip of a nose of the user, and a mouth of the user; and
detecting the magnitude of the emotional state of the user based on the identified facial landmarks and a set of emotional classification rules.

6. The method of claim 5,
wherein determining the magnitude of the emotional state of the user comprises:
determining facial attributes of the user based on one or more of the identified facial landmarks, the facial attributes including measurements of one or more of the identified facial landmarks or between two or more of the identified facial landmarks; and
determining the magnitude of the emotional state of the user based on the determined facial attributes.

7. The method of claim 1,
wherein causing the graphical representation of the user to be rendered using the selected rule comprises generating an avatar representation of the user for display.

8. The method of claim 1,
wherein receiving the configuration instruction includes receiving an input from the user to suppress the first emotional state or to modify a magnitude of the first emotional state of the user from a default or previously received configuration instruction.

9. The method of claim 1,
wherein causing the graphical representation of the user to be rendered using the selected rule comprises rendering a synthetic image of the user to communicate the displayed emotional state using a facial animation model, wherein the facial animation model includes a training model of the user.

10. The method of claim 1,
wherein the sentiment analysis comprises a neural network.

11. The method of claim 1,
wherein the facial animation rules comprise one or more weights for a deep reinforcement machine-learning model.

12. A system for displaying an emotional states of a user using a graphical representation of the user, the graphical representation having a displayed emotional state, comprising:
one or more processors; and
a memory storing computer-executable instructions that, when executed, cause the one or more processors to control the system to perform operations comprising:
receiving a configuration instruction for a first emotional state, the configuration instruction specifying that the first emotional state is to be modified;
detecting, based on a received image of the user, an emotional state of the user and a magnitude of the detected emotional state of the user using sentiment analysis;
determining a modified emotional state corresponding to the detected emotional state for the graphical representation of the user based upon the detected emotional state of the user and the configuration instruction, the modified emotional state of the graphical representation modifying the detected emotional state by being a different emotional state or a change in the magnitude of the detected emotional state;
selecting a rule from a set of facial animation rules based upon the modified emotional state and the detected emotional state of the user, the rule specifying instructions for rendering the graphical representation of the user that has a facial expression that is mapped to the modified emotional state; and
causing the graphical representation of the user to be rendered using the selected rule.

13. The system of claim 12,
wherein determining the modified emotional state for the graphical representation of the user based upon the detected emotional state of the user and the configuration instruction comprises determining a different emotional state than the detected emotional state, including one of:
a previously displayed emotional state of the user preceding the determined emotional state;
a neutral emotional state; or
a prespecified replacement emotional state specified according to the configuration instruction.

14. The system of claim 13,
wherein receiving the configuration instruction for the first emotional state includes receiving the replacement emotional state.

15. The system of claim 12,
wherein the first emotional state is one of a set of emotional states comprising happiness, sadness, neutral, anger, contempt, disgust, surprise, and fear, and
wherein detecting the magnitude of the detected emotional state of the user includes determining a score for the set of emotional states based on the received image of the user and selecting the detected emotional state from the set of emotional states having a highest score as the detected emotional state of the user.

16. The system of claim 12,
wherein detecting the magnitude of the detected emotional state of the user using sentiment analysis includes:
receiving the image including a face of the user;
identifying facial landmarks of the face of the user from the received image, including locations of pupils of the user, a tip of a nose of the user, and a mouth of the user; and
detecting the magnitude of the emotional state of the user based on the identified facial landmarks and a set of emotional classification rules.

17. The system of claim 16,
wherein determining the magnitude of the emotional state of the user comprises:
determining facial attributes of the user based on one or more of the identified facial landmarks, the facial attributes including measurements of one or more of the identified facial landmarks or between two or more of the identified facial landmarks; and
determining the magnitude of the emotional state of the user based on the determined facial attributes.

18. The system of claim 12,
wherein causing the graphical representation of the user to be rendered using the selected rule comprises generating an avatar representation of the user for display.

19. The system of claim 12,
wherein receiving the configuration instruction includes receiving an input from the user to suppress the first emotional state or to modify a magnitude of the first emotional state of the user from a default or previously received configuration instruction.

20. A system for displaying an emotional states of a user using a graphical representation of the user during a communication session, the graphical representation having a displayed emotional state, comprising:
means for receiving a configuration instruction for a first emotional state, the configuration instruction specifying that the first emotional state is to be modified;
means for detecting, based on a received image of the user during the communication session, an emotional state of the user and a magnitude of the detected emotional state of the user using sentiment analysis;
means for determining a modified emotional state for the graphical representation of the user based upon the detected emotional state of the user and the configuration instruction, the modified emotional state of the graphical representation modifying the detected emotional state by being a different emotional state or a change in the magnitude of the detected emotional state;
means for selecting a rule from a set of facial animation rules based upon the modified emotional state and the detected emotional state of the user, the rule specifying instructions for rendering the graphical representation of the user that has a facial expression that is mapped to the modified emotional state; and
means for causing the graphical representation of the user to be rendered using the selected rule.
